(19) United States
(12) Patent Application Publication
(10) Pub. No.: US 2026/0224880 A1
(43) Pub. Date: Aug. 6, 2026

(54) CATHETER SYSTEM FOR PULSATILE FLOW VISUALIZATION AND BLOOD CONTAINMENT

(71) Applicant: Becton, Dickinson and Company, Franklin Lakes, NJ (US)

(72) Inventors: Curtis H. Blanchard, Herriman, UT (US); Weston F. Harding, Lehi, UT (US); Thomas Solosko, Draper, UT (US); Ralph L. Sonderegger, Farmington, UT (US); Daniel Blanchard, Bountiful, UT (US)

(21) Appl. No.: 19/047,404

(22) Filed: Feb. 6, 2025

(51) Int. Cl. A61M 39/20; A61M 39/10; A61M 25/06

(52) U.S. Cl. CPC A61M 39/20; A61M 39/10; A61M 25/0693; A61M 2039/1072; A61M 2039/1077; A61M 2039/205

(57) ABSTRACT

A catheter system may include a vent plug coupled to a connector and configured to vent air from an extension tubing and through a septum when a button causes the septum to open. A catheter system may include a button having a venting pathway with a first end and a second end laterally offset from the first end. When the button is depressed, air may vent through the venting pathway. A catheter system may include a cover configured to rotate or slide with respect to an extension tubing to allow air to pass through a membrane filter. A catheter system may include a lock piece configured to prevent removal of a first cap before a second cap. A catheter system may include a vent plug having a venting pathway and configured to move from a first position to a second position to allow venting through the venting pathway.

### BACKGROUND

Catheter assemblies are a ubiquitous part of hospital care and are used as a main access point to draw blood, infuse medications, and assess bodily function. Typically, catheter assemblies include a catheter and a catheter adapter having a lumen in fluid communication with the catheter. Upon insertion of the catheter into the vasculature of a patient, the patient’s blood flows freely though the catheter and into the lumen of the catheter adapter. This blood flow is termed “flashback” and is desirable to ensure proper insertion of the catheter.

As its name implies, a catheter that is “over-the-needle” may be mounted over an introducer needle having a sharp distal tip. The catheter and the introducer needle can be inserted at a shallow angle through the skin and into the vasculature of the patient. In order to verify proper placement of the introducer needle and the catheter in the vasculature, a clinician may confirm there is flashback of blood. After placement of the introducer needle within the vasculature has been confirmed, the clinician may remove the introducer needle, leaving the catheter in place in the vasculature for future blood draw or fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a catheter system, as well as related devices and methods. In order to verify proper placement of the catheter system in the vasculature, a clinician may confirm there is flashback of blood. When the catheter is inserted into an artery, the flashback of blood may include pulsatile blood flow, according to embodiments of the present disclosure. Pulsatile blood flow in an arterial catheter assembly refers to the rhythmic flow of blood within the arterial catheter assembly that mirrors the natural pulsations of the heart. This occurs because arteries carry blood under higher pressure than veins, driven by the contractions and relaxations of the heart during systole (when the heart pumps blood) and diastole (when the heart rests between beats). The pulsatile nature of arterial blood flow allows clinicians to connect the arterial catheter assembly to a monitoring system that can measure and display dynamic parameters in real time.

When the catheter system of the present disclosure is positioned within an artery of a patient, high pressure from arterial blood flow may push against walls of the particular catheter system, including an attached extension tubing. In some embodiments, the catheter system of the present disclosure includes an extension set having an extension tubing that is clear, and the pressure waves can cause the blood to visibly “pulse” in rhythm with the patient’s heartbeat inside the extension tubing. Importantly, in some embodiments, the catheter system of the present disclosure facilitates visual monitoring of pulsatile blood pressure continuously during catheter insertion after arterial penetration while at a same time providing blood containment.

According to a first set of embodiments, a catheter system may include a catheter adapter, which may include a distal end, a proximal end, and a side port in between the distal end of the catheter adapter and the proximal end of the catheter adapter. The catheter system may include a catheter extending distally from the distal end of the catheter adapter.

The catheter system may include an extension tubing having a distal end and a proximal end. The distal end of the extension tubing may be coupled to the side port. The catheter system may include a connector coupled to the proximal end of the extension tubing. The connector may include a distal end, a proximal end, a lumen extending through the distal end of the connector and the proximal end of the connector, and a window disposed between the distal end of the connector and the proximal end of the connector. The lumen may be in fluid communication with the extension tubing.

The catheter system may include a septum positioned within the lumen. The septum may include a slit that is biased in a closed position to prevent fluid flow through the lumen. The catheter system may include a button disposed within the window. When the button is pressed, the button may cause the slit of the septum to open thereby creating a fluid pathway through the lumen.

The catheter system may include a vent plug coupled to the proximal end of the connector. The vent plug may be configured to vent air from the extension tubing and through the septum when the button causes the slit of the septum to open. The vent plug may include a venting pathway extending through the vent plug. The vent plug may include a membrane filter disposed within the venting pathway and configured to pass air but not blood.

According to a second set of embodiments, a catheter system may include a catheter adapter, which may include a distal end, a proximal end, and a side port in between the distal end of the catheter adapter and the proximal end of the catheter adapter. The catheter system may include a catheter extending distally from the distal end of the catheter adapter. The catheter system may include an extension tubing having a distal end and a proximal end. The distal end of the extension tubing may be coupled to the side port.

The catheter system may include a connector coupled to the proximal end of the extension tubing. The connector may include a distal end, a proximal end, a lumen extending through the distal end of the connector and the proximal end of the connector, and a slot disposed between the distal end of the connector and the proximal end of the connector. The lumen may be in fluid communication with the extension tubing.

The catheter system may include a button disposed within the slot. The button may include a venting pathway extending through the button. The venting pathway may include a first end and a second end laterally offset from the first end. The venting pathway may be configured to pass air and not blood. When the button is in a raised position, the first end may be blocked from fluid communication with the lumen, and the second end may be in fluid communication with ambient air. When the button is depressed, the first end may be disposed within the lumen, and the second end may be in fluid communication with ambient air such that air vents through the venting pathway from the lumen into the ambient air.

The connector may include a spring disposed within the lumen and configured to return the button from the depressed position to the raised position. The catheter system may include a cap sealing the proximal end of the connector.

According to a third set of embodiments, a catheter system may include a catheter adapter, which may include a distal end, a proximal end, and a side port in between the distal end of the catheter adapter and the proximal end of the catheter adapter. The catheter system may include a catheter extending distally from the distal end of the catheter adapter. The catheter system may include an extension tubing having a distal end, a proximal end, and a window disposed between the distal end of the extension tubing and the proximal end of the extension tubing. The distal end of the extension tubing may be coupled to the side port. The window may include a membrane filter therein. The membrane filter may be configured to pass air and not blood thereby preventing blood from exiting the window.

The catheter system may include a connector coupled to the proximal end of the extension tubing. The connector may include a distal end, a proximal end, a lumen extending through the distal end of the connector and the proximal end of the connector. The lumen may be in fluid communication with the extension tubing.

The catheter system may include a cover configured to cover the window to block fluid communication between a lumen of the extension tubing and the ambient air. The cover may be configured to be rotated or slid with respect to the extension tubing to uncover the window and allow the air to pass through membrane filter from the lumen of the extension tubing to the ambient air.

The cover may include a snap-on cover extending partially around a circumference of the extension tubing and having another window. The other window may include an open space without a membrane filter therein. When the snap-on cover is in a first position, the window and the other window may be misaligned such that the cover prevents the air from venting through the membrane filter into ambient air. The snap-on cover may be configured to rotate around the extension tubing to a second position. When the snap-on cover is in the second position, the window and other window may be aligned such that air is configured to vent through the membrane filter into the ambient air.

The cover may include an annular body extending around an entirety of a circumference of the extension tubing and having another window. When the annular body is in a first position, the window and the other window may be misaligned such that the cover prevents the air from venting through the membrane filter into ambient air. The annular body may be configured to rotate around the extension tubing to a second position. When the annular body is in the second position, the window and other window may be aligned such that air is configured to vent through the membrane filter into the ambient air.

According to a fourth set of embodiments, a catheter system may include a catheter adapter, which may include a distal end, a proximal end, and a side port in between the distal end of the catheter adapter and the proximal end of the catheter adapter. The catheter system may include a catheter extending distally from the distal end of the catheter adapter. The catheter system may include an extension tubing having a distal end and a proximal end. The distal end of the extension tubing may be coupled to the side port.

The catheter system may include a connector coupled to the proximal end of the extension tubing. The connector may include a distal end, a proximal end, and a lumen extending through the distal end of the connector and the proximal end of the connector. The lumen may be in fluid communication with the extension tubing.

The catheter system may include a first cap threadedly coupled to the proximal end of the connector. The first cap may include a male luer disposed within the proximal end of the connector and a venting pathway extending through the male luer. The venting pathway may include a membrane filter to pass air and not blood and/or may be sized and configured to pass air and not blood. The male luer of the first cap may be short such that the male luer does not interfere with the membrane filter.

The catheter system may include a lock piece having an aperture therethrough. The lock piece may prevent the first cap from being unthreaded from the proximal end of the connector. The catheter system may include a second cap extending through the aperture and forming a seal inside the male luer. The lock piece may be configured to be removed from the catheter system with the second cap before the first cap can be unthreaded from the proximal end of the connector.

The first cap may include threading that is segmented and disposed on opposing portions of the first cap spaced apart by a first gap and second gap. The lock piece may include a first arm disposed within the first gap and a second arm disposed within the second gap thereby preventing the first cap from rotating and being unthreaded from the proximal end of the connector.

According to a fifth set of embodiments, a catheter system may include a catheter adapter, which may include a distal end, a proximal end, and a side port in between the distal end of the catheter adapter and the proximal end of the catheter adapter. The catheter system may include a catheter extending distally from the distal end of the catheter adapter. The catheter system may include an extension tubing having a distal end and a proximal end. The distal end of the extension tubing may be coupled to the side port.

The catheter system may include a connector coupled to the proximal end of the extension tubing. The connector may include a distal end, a proximal end, and a lumen extending through the distal end of the connector and the proximal end of the connector. The lumen may be in fluid communication with the extension tubing.

The catheter system may include a vent plug coupled to the catheter system and movable between a first position and a second position when coupled to the catheter system. The vent plug may include a venting pathway extending through the vent plug. The venting pathway may include a membrane filter to pass air and not blood and/or may be sized and configured to pass air and not blood. In response to the vent plug being moved from the first position to the second position, the venting pathway may be in fluid communication with the lumen such that air vents through the venting pathway from the lumen into the ambient air.

The catheter system may include another connector coupled to the proximal end of the connector and having another lumen. A septum may be disposed within the other connector. The catheter system may include a housing coupled to the proximal end of the other connector. The vent plug may be movable within the housing from the first position and the second position. The septum may include a slit that is biased in a closed position to prevent fluid flow through the other lumen. In response to the vent plug being moved from the first position to the second position, the vent plug may open the slit of the septum such that the venting pathway is in fluid communication with the lumen and air vents through the venting pathway from the lumen into the ambient air.

The catheter system may include the other connector coupled to the proximal end of the connector and having another lumen. The catheter system may include a septum disposed within the other connector. The vent plug may be threaded with respect to the other connector from the first position and the second position. The septum may include a slit that is biased in a closed position to prevent fluid flow through the other lumen. In response to the vent plug being threaded with respect to the other connector from the first position to the second position, the vent plug may open the slit of the septum such that the venting pathway is in fluid communication with the lumen and air vents through the venting pathway from the lumen into the ambient air.

The vent plug may be coupled to the proximal end of the connector. The catheter system may include a septum disposed within the lumen and a septum actuator disposed within the lumen. The vent plug may be threaded with respect to the other connector from the first position and the second position. In response to the vent plug being threaded with respect to the connector from the first position to the second position, the vent plug may push the septum actuator distally to open the septum such that the venting pathway is in fluid communication with the lumen and air vents through the venting pathway from the lumen into the ambient air.

The vent plug may be coupled to the proximal end of the connector having the lumen. The vent plug may be unthreaded with respect to the connector from the first position and the second position. A portion of the vent plug configured to insert into the lumen may be oblong. In response to the vent plug being moved from the second position to the first position, the portion may be configured to compress and seal the lumen.

The vent plug may be coupled to the proximal end of the connector. The vent plug may be unthreaded with respect to the connector from the first position and the second position. The vent plug may include a male luer having multiple grooves. In response to the vent plug being threaded with respect to the connector from the second position to the first position, the grooves may be compressed or pressure within the grooves increases such that the venting pathway is blocked.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a perspective view of an example catheter system, according to some embodiments;

FIG. 2A is a perspective view of an example catheter system having an example connector with an example flow control button and example vent plug, according to some embodiments;

FIG. 2B is a perspective view of the connector of FIG. 2A in an example closed state, according to some embodiments;

FIG. 2C is a cross-sectional side view of the connector of FIG. 2A in the closed state, according to some embodiments;

FIG. 2D is a cross-sectional top view of the connector of FIG. 2A in the closed state, according to some embodiments;

FIG. 2E is a cross-sectional view of the connector in the closed state along the line 2E-2E of FIG. 2D, according to some embodiments;

FIG. 2F is a perspective rear view of the connector in the closed state along the line 2F-2F of FIG. 2D, according to some embodiments;

FIG. 2G is a cross-sectional view of the connector in the closed state along the line 2G-2G of FIG. 2F, according to some embodiments;

FIG. 2H is a cross-sectional side view of the connector of FIG. 2A in an example opened state, according to some embodiments;

FIG. 2I is a cross-sectional top view of the connector of FIG. 2A in the opened state, according to some embodiments;

FIG. 2J is a cross-sectional view of the connector in the opened state along the line 2J-2J of FIG. 2I, according to some embodiments;

FIG. 2K is a perspective rear view of the connector in the opened state along the line 2K-2K of FIG. 2I, according to some embodiments;

FIG. 2L a is a cross-sectional view of the connector in the opened state along the line 2L-2L of FIG. 2K, according to some embodiments;

FIG. 3A is a cross-sectional view of another example connector having an example flow control button, illustrating the connector is an example closed state, according to some embodiment;

FIG. 3B is a cross-sectional view of the connector of FIG. 3A, illustrating the connector in an example open state, according to some embodiments;

FIG. 4A is a cross-sectional view of an example snap-on cover coupled to an example extension tubing, according to some embodiments;

FIG. 4B is a perspective view of the snap-on cover of FIG. 4A, according to some embodiments;

FIG. 4C is a perspective view of another example cover, according to some embodiments;

FIG. 4D is a perspective view of the snap-on cover of FIG. 4A with a membrane filter within an example other window, according to some embodiments;

FIG. 5 is a cross-sectional view of an example catheter assembly, according to some embodiments;

FIG. 6A is a cross-sectional view of an example vent plug assembly coupled to an example connector, illustrating an example vent plug in an example first position, according to some embodiments;

FIG. 6B is a cross-sectional view of the vent plug assembly of FIG. 6A coupled to the connector, illustrating the vent plug in an example second position, according to some embodiments;

FIG. 7A is a cross-sectional view of another example vent plug assembly coupled to an example connector, illustrating an example vent plug in an example first position, according to some embodiments;

FIG. 7B is a cross-sectional view of the vent plug assembly of FIG. 7A coupled to the connector, illustrating the vent plug in an example second position, according to some embodiments;

FIG. 8A is a cross-sectional view of another example vent plug assembly coupled to an example connector, illustrating an example vent plug in an example first position, according to some embodiments;

FIG. 8B is a cross-sectional view of the vent plug assembly of FIG. 8A coupled to the connector, illustrating the vent plug in an example second position, according to some embodiments;

FIG. 9A is a perspective view of an example vent plug assembly coupled to an example connector, according to some embodiments;

FIG. 9B is a cross-sectional view along the line 9B-9B of FIG. 9A, illustrating the vent plug assembly coupled to the connector, according to some embodiments;

FIG. 9C is a perspective view of an example first cap of the vent plug assembly of FIG. 9A, illustrating the first cap coupled to the connector, according to some embodiments;

FIG. 9D is a perspective view of an example locking cap of the vent plug assembly of FIG. 9A, according to some embodiments;

FIG. 9E is a cross-sectional view along the line 9E-9E of FIG. 9A, illustrating the vent plug assembly coupled to the connector, according to some embodiments;

FIG. 10A is a perspective view of another example vent plug having example grooves, according to some embodiments;

FIG. 10B is a cross-sectional view of the vent plug of FIG. 10A, according to some embodiments;

FIG. 10C is a cross-sectional view of the vent plug of FIG. 10A coupled to an example connector, according to some embodiments; and

FIG. 10D is a lower perspective view of another example vent plug, according to some embodiments.

### DESCRIPTION OF EMBODIMENTS

For clarity it is to be understood that the word “proximal” refers to a direction relatively closer to a clinician using the catheter system described in the present disclosure, and the word “distal” refers to a direction relatively further from the clinician using the catheter system described in the present disclosure. For example, the end of a catheter placed within the body of a patient is considered a distal end of the catheter, while the catheter end remaining outside the body is considered a proximal end of the catheter. Also, the words “including,” “has,” and “having,” as used in the present disclosure, including the claims, shall have the same meaning as the word “comprising.”

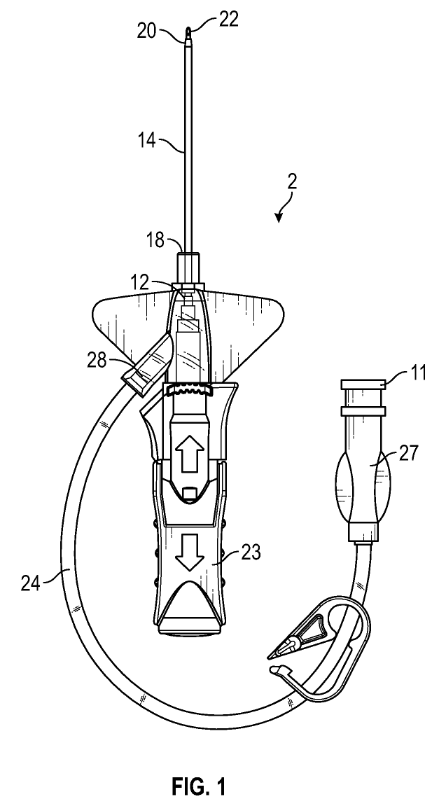

FIG. 1 illustrates a catheter system 2, according to some embodiments. In some embodiments, the catheter system 2 may include a catheter adapter 3 having a proximal end 4, a distal end 5, and a side port 6 in between the distal end 5 of the catheter adapter 3 and the proximal end 4 of the catheter adapter 3. In some embodiments, the catheter system 2 may include a catheter 7 extending distally from the distal end 5 of the catheter adapter 3.

In some embodiments, the catheter system 2 may include an extension set, which may include an extension tubing 8 having a distal end and a proximal end. In some embodiments, the distal end of the extension tubing 8 may be coupled to the side port 6. In some embodiments, the catheter system 2 may include a connector 9 coupled to the proximal end of the extension tubing 8. In some embodiments, the connector 9 may include a distal end, a proximal end, and a lumen extending through the distal end of the connector 9 and the proximal end of the connector 9. In some embodiments, the lumen of the connector 9 may be in fluid communication with the extension tubing 8, which is in fluid communication with the catheter 7. In some embodiments,

In some embodiments, the catheter system 2 may include a cap 11 that may be coupled to the connector 9. In some embodiments, the cap 11 may be unvented or not allow venting therethrough. Thus, the catheter system 2 may allow visualization of pulsatile blood flow when the connector 9 is capped using the cap 11. In some embodiments, when an artery is accessed by the catheter system 2, arterial pressure pressurizes the air in the extension tubing 8, producing a visible blood pulse within the extension tube 8 throughout the catheterization procedure. In some embodiments, upon removing the cap 11 to attach an IV line to the connector 9, arterial blood may flow through the connector 9. Thus, to avoid or reduce blood exposure, a clinician is often required to manually stop the blood flow with a finger or quickly attach the IV line. This may be difficult and can result in error and subsequent blood leakage from the connector 9. Blood leakage from the connector 9 when attaching the IV line poses a biohazard risk, potentially exposing clinicians and patients to bloodborne pathogens, while also contaminating gloves, bedding, and surrounding surfaces.

In some embodiments, after the pulsatile blood flow is observed and before removing the cap 11, a needle assembly 13, including an introducer needle 15 extending through the catheter 7 and used to penetrate the skin and artery, may be removed from the catheter system 2. In some embodiments, the catheter system 2 may be an “over-the-needle” catheter system having the introducer needle 15 extending through the catheter 7.

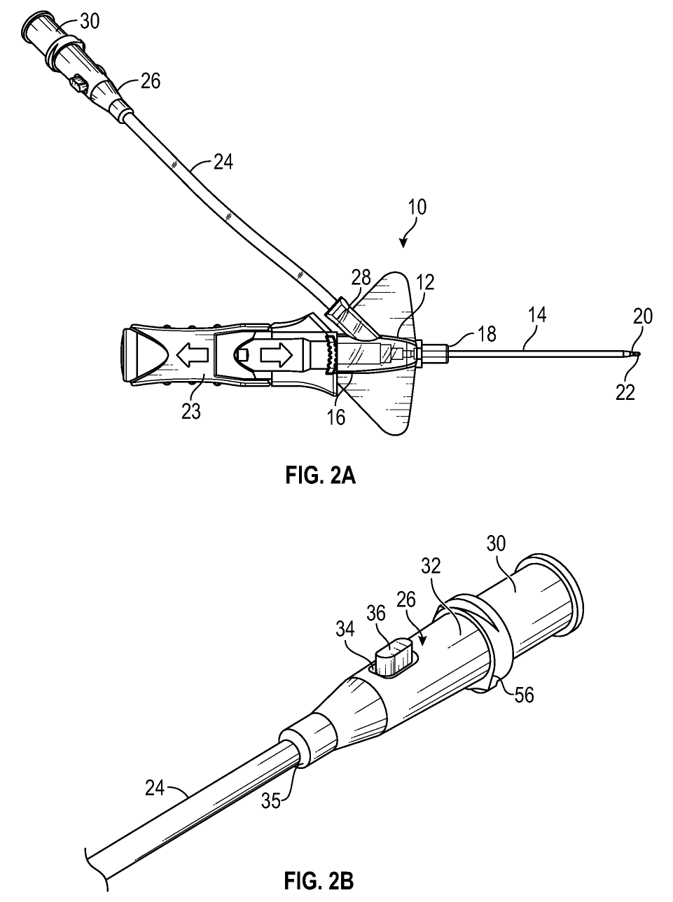

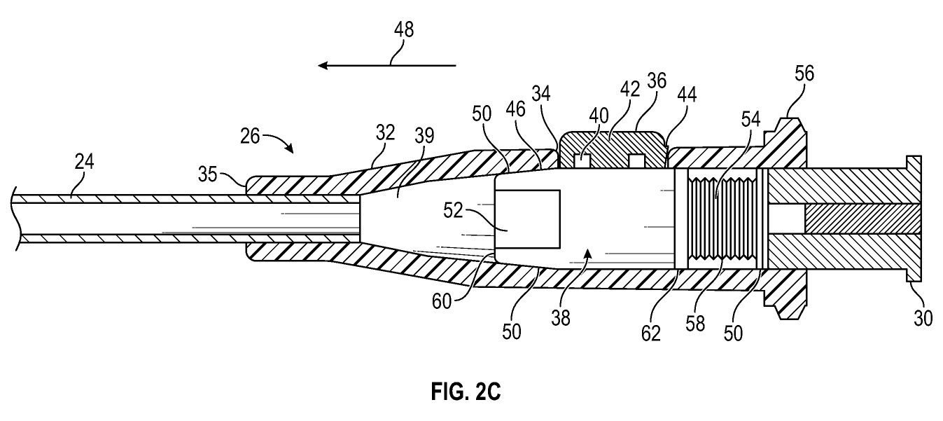

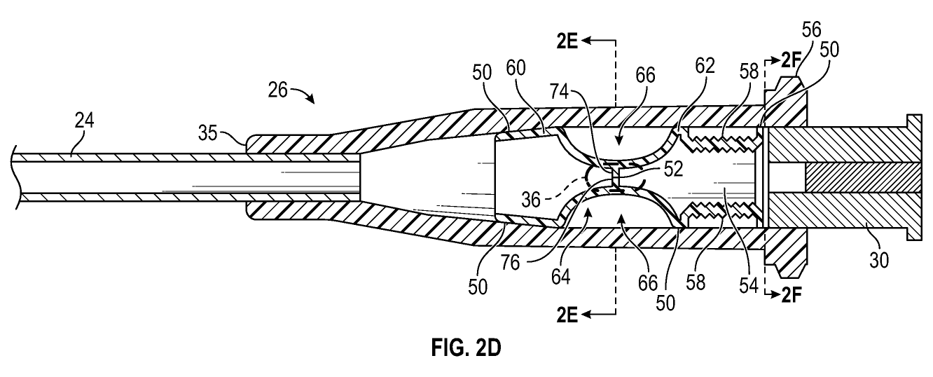

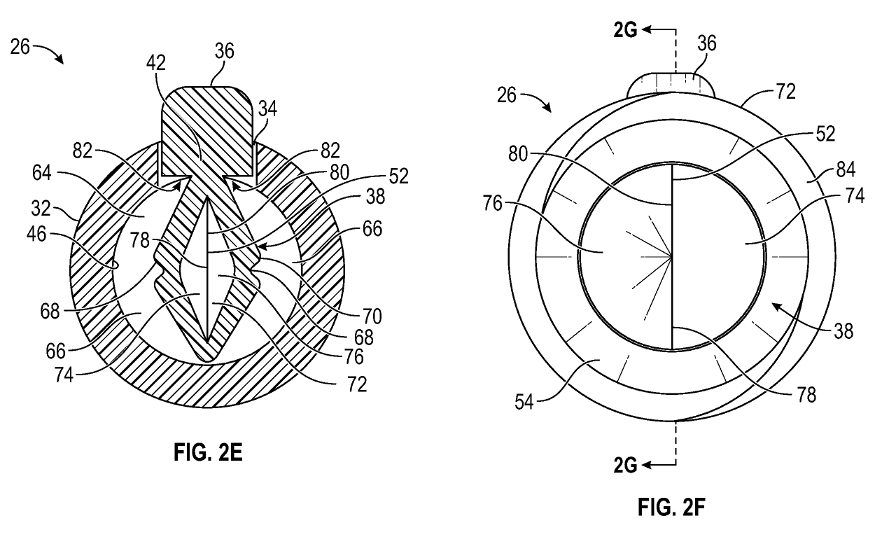

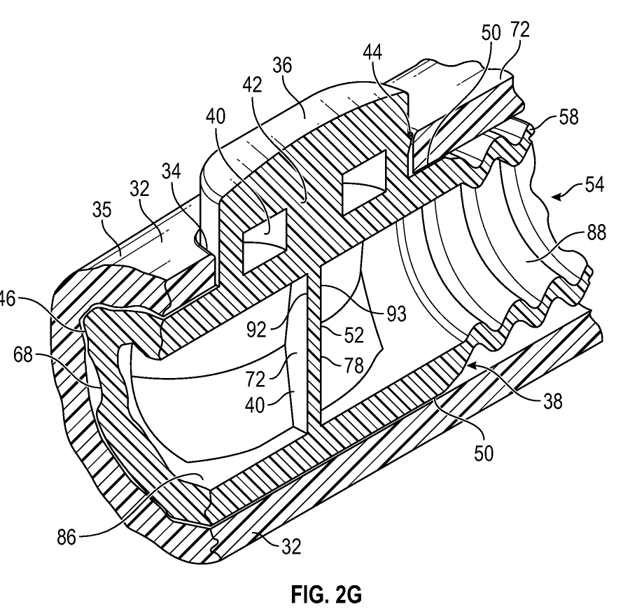

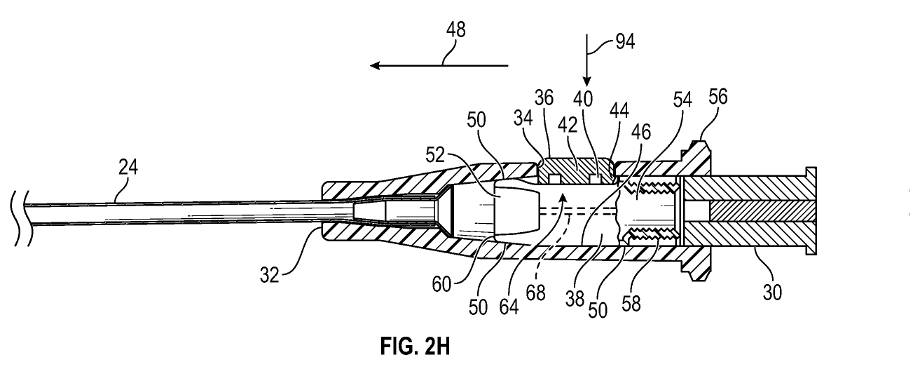

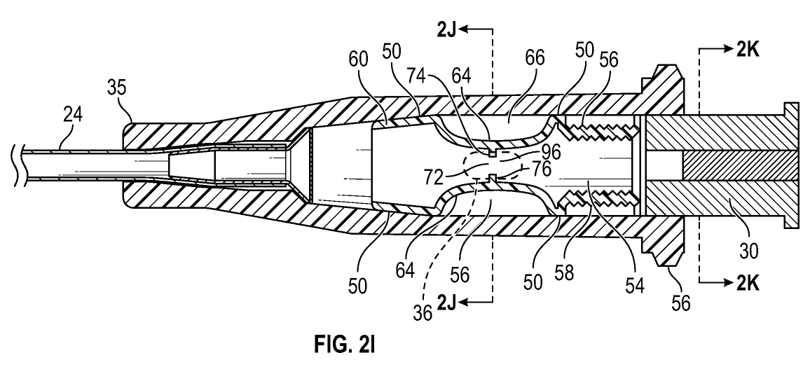

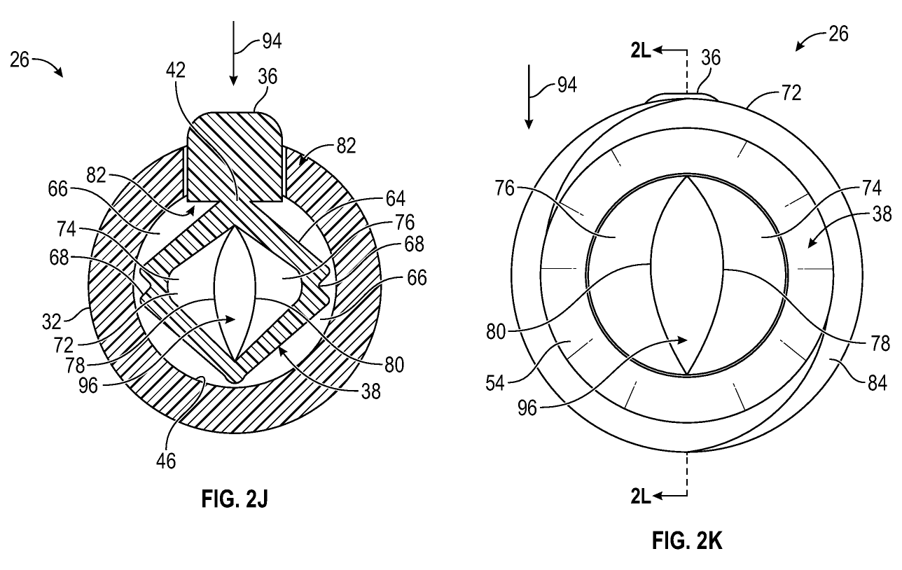

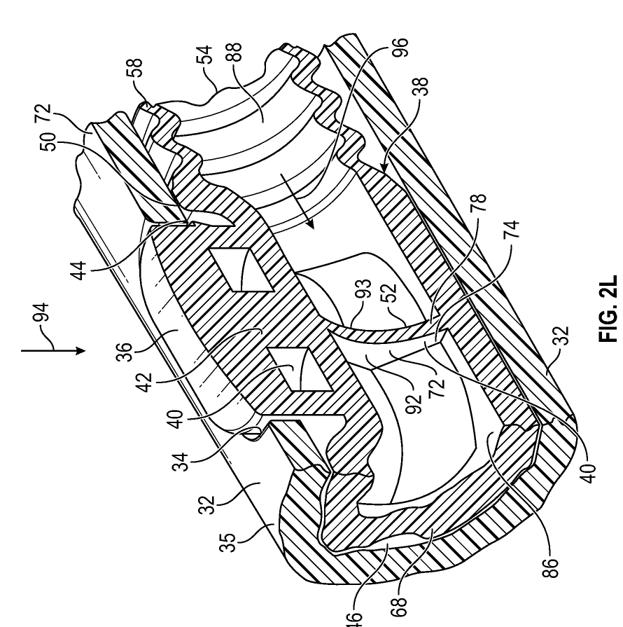

Referring now to FIG. 2A, a catheter system 10 is illustrated, according to some embodiments. In some embodiments, the catheter system 10 may include a catheter assembly having a catheter adapter 12 and a catheter 14. In some embodiments, the catheter adapter 12 may include a generally tubular body. In some embodiments, the catheter adapter 12 may include a rigid or semi-rigid polymer material, such as, for example, polystyrene or polypropylene. In some embodiments, the catheter adapter 12 may include a proximal end 16 and a distal end 18. In some embodiments, the catheter system 10 may be similar or identical to the catheter system 2 in terms of one or more components and/or operation.

In some embodiments, the catheter 14 may extend from the distal end 18 of the catheter adapter 12. In some embodiments, the catheter 14 may include flexible or semi-flexible tubing that may be inserted into vasculature of a patient, such as an artery. In some embodiments, the catheter 14 may be generally tubular with an inner lumen configured to permit the passing of an introducer needle 20 having a sharp distal tip 22. In some embodiments, a needle assembly 23 may be coupled to the proximal end 16 of the catheter adapter 12, and the introducer needle 20 of the needle assembly 23 may extend through the catheter 14 to aid in introduction of the catheter 14 into the vasculature of the patient. In this way, the introducer needle 20 may pierce the skin of the patient, and the catheter 14 may be inserted into the patient through the opening created by the introducer needle 20.

In some embodiments, the catheter system 10 may include an extension tubing 24 in fluid communication with the catheter adapter 12 and the catheter 14. In some embodiments, the catheter system 10 may include a connector 26 proximal to the extension tubing 24 and in fluid communication with the extension tubing 24. In some embodiments, the catheter adapter 12 may include a side port 28 disposed between the distal end 18 and the proximal end 16. In some embodiments, the extension tubing 24 may include a distal end integrated with the side port 28.

In some embodiments, the extension tubing 24 may include a proximal end integrated with the connector 26. In some embodiments, the extension tubing 24 may be flexible and configured to curve and straighten. In some embodiments, the extension tubing 24 may provide an increased distance between the connector 26 and the catheter adapter 12 to reduce a risk of disturbing the catheter 14 within the vasculature when an IV line, a blood collection device, or other device is coupled to the connector 26. In some embodiments, a clamp (not illustrated) may be disposed on the extension tubing 24. In some embodiments, the catheter system 10 may include a vent plug 30.

Referring now to FIG. 2B, in some embodiments, the connector 26 may include a connector body 32 having a window 34. In some embodiments, the connector 26 may include a distal end 35 and a proximal end 56. In some embodiments, the proximal end 56 may include a female luer. In some embodiments, the window 34 may include an opening in the surface of the connector body 32 and may provide an opening to house a flow control button 36. In some embodiments, the window 34 may further include a system for sealing the window 34 against fluids within the connector 26. For example, a gasket or sealant may be incorporated between the window 34 and the flow control button 36 such that a fluid is prevented from penetrating the interface of the window 34 and the flow control button 36. In some embodiments, the flow control button 36 may form a part of the connector body 32. In some embodiments, a fluid within the connector 26 will be contained within the connector body 32.

Referring now to FIG. 2C, the connector 26 of FIG. 2B is illustrated in cross-sectional side view, according to some embodiments. As previously discussed, in some embodiments, the connector body 32 may include the window 34 for providing an opening to house the flow control button 36. In some embodiments, the flow control button 36 may include an extended portion of a valve member or a septum 38. In some embodiments, the septum 38 may be disposed within a lumen 39 of the connector 26. In some embodiments, the primary function of the septum 38 is to control the flow of a fluid through the connector 26.

In some embodiments, the flow control button 36 may be modified to include a system of channels 40 that include the width of the flow control button 36 and are located between the flow control button 36 and the septum 38. In some embodiments, the channels 40 may be formed by removing a portion of the material of the flow control button 36 such that the channels 40 may be formed perpendicular to the length of the flow control button 36. In some embodiments, by removing a portion of the flow control button 36 material, the force of the flow control button 36 may be focused towards a middle section 42 of the flow control button 36. As such, in some embodiments, the channels 40 may allow a user to apply pressure to a focused portion of the septum 38 by generally depressing the flow control button 36. As will be described in detail below, in some embodiments, the focused force of the flow control button 36 allows for the proper and accurate opening of a slit 52 through the septum 38.

In some embodiments, the flow control button 36 may be further modified to include a catch 44. In some embodiments, the catch 44 may include a wedged extension of the button whereby the catch 44 is configured to interact with an edge of the window 34 of connector 26. For example, a user may depress the flow control button 36 such that the catch 44 passes to the inside of the window 34 opening. As such, the catch 44 may engage an inner surface 46 of the connector 26 and prevent the flow control button 36 from returning to a released position. In this manner, the flow control button 36 may continue to be depressed without requiring contact by the user. In some embodiments, the catch 44 may be disengaged from the inner surface 46 of the connector 26 by simultaneously depressing the button and biasing the button in a distal or forward direction 48. In some embodiments, the flexibility of the flow control button 36 thereby permits the button to advance in the forward direction 48 to allow the catch 44 to disengage and return through the window 34 opening to a released position.

In some embodiments, the design of both the septum 38 and the flow control button 36 may be adapted to control the flow of a fluid through the catheter system 10. In some embodiments, the cap 11 of the catheter system 2 that is unvented may not be needed to provide backpressure to visualize the pulsatile blood flow in the extension tubing 24. Instead, the septum 38 may be closed to provide the backpressure. In some embodiments, after the pulsatile blood flow is observed by the clinician in the extension tubing 24 indicating the catheter 14 is inserted into the artery, the clinician can apply pressure to the flow control button 36 and thereby apply pressure to the septum 38 to open the septum 38. Opening the septum 38 may allow venting of air through the vent plug 30 and blood to travel through the extension tubing 24 and to the connector 26 prior to attachment of the IV line or another device to the connector 26.

In some embodiments, after the septum 38 is opened and blood is sufficiently advanced, the clinician can release the pressure on the flow control button 36 to close the septum 38, so that the clinician can then remove the vent plug 30 and attach the IV line or other device to the proximal end 56 of the connector 26. In some embodiments, the IV line or other device may include a male luer that penetrates the septum 38 to allow fluid to travel through the septum 38. In some embodiments, after the IV line or other device is connected to the connector 26, the clinician may press the button hard enough for the catch 44 to pass to the inside of the window 34 and engage the inner surface 46 of the connector 26, locking the septum 38 in an opened state for infusion, blood monitoring, etc.

In some embodiments, the septum 38 may be designed to include an interface 50 with the inner surface 46 of the connector 26 such that a fluid-tight interface is created. Therefore, a fluid, such as the blood of the patient, may be prevented from flowing past a distal end 60 of the septum 38. In some embodiments, the interface 50 may include a pressure fitting, an O-ring, a gasket or a portion of the septum 38. In some embodiments, the interface 50 may also prevent a fluid from leaking through the window 34. This is accomplished by configuring the interface 50 to sufficiently contact the inner surface 46, thereby preventing passage of a fluid between the septum 38 and the inner surface 46.

In some embodiments, the septum 38 may be further modified to include a slit 52 thereby providing a pathway through the septum 38. In some embodiments, the dimensions of the slit 52 may vary depending upon the needs of the catheter system 10. In some embodiments, the septum 38 and the slit 52 may be designed such that the slit 52 is biased in a closed state. In some embodiments, the slit 52 may be biased open by depressing the flow control button 36, as discussed in greater detail below.

In some embodiments, the septum 38 may further include a docking portion 54 located within a proximal end 56 of the connector 26. In some embodiments, the docking portion 54 may include a lateral extension of the septum 38. In some embodiments, the docking portion 54 may further include a ribbed section 58. In some embodiments, the ribbed section 58 may include a series of raised, annular ridges circumscribing the inner and outer surfaces of the docking portion 54. In some embodiments, the ribbed section 58 may permit the docking portion 54 of the septum 38 to be compressed in the forward direction 48 as a probe is inserted. In some embodiments, the raised, annular ridges of the ribbed section 58 may permit the ribbed section 58 to compress in an accordion-like fashion thereby permitting continued insertion of the IV line or other device that includes the male luer. In some embodiments, the compressed position of the ribbed section 58 may be maintained by the inserted the IV line or other device that includes the male luer. In some embodiments, upon removal of the IV line or other device that includes the male luer, the compressed ribbed section 58 may be returned to its original, unfolded configuration. In some embodiments, the compressed ribbed section 58 may exert a recoil force on the IV line or other device that includes the male luer. As such, the negative force required to remove the inserted probe is reduced due to the positive recoil force of the compressed ribbed section 58.

Importantly, in some embodiments, the vent plug 30 may include a short luer that cannot actuate the septum 38. Thus, the septum 38 can be closed when the vent plug 30 is inserted to allow visualization of the pulsatile blood flow within the extension tubing 24. In some embodiments, when the vent plug 30 is secured to the proximal end 56 of the connector 26, the vent plug 30 may not contact the septum 38 and/or the vent plug 30 may not compress the ribbed section 58 or docking portion 54.

In some embodiments, the vent plug 30 may be configured to vent air from the extension tubing 24 and through the septum 38 when the flow control button 36 causes the slit 52 of the septum 38 to open. In some embodiments, the vent plug 30 may include a venting pathway 59 extending through the vent plug 30. In some embodiments, the vent plug may include a membrane filter 61 disposed within the venting pathway to pass air and not blood and/or may be sized and configured to pass air but not blood. In some embodiments, the venting pathway that is sized or configured to pass air but not blood may include a small hole or gap that allows air to pass and not liquid.

In some embodiments, the membrane filter 61 may include a hydrophobic membrane, such as, for example, polytetrafluoroethylene (PTFE) to provide hydrophobic properties and chemical resistance and facilitate airflow. In some embodiments, the membrane filter 61 may include polyethylene, polypropylene, or another plastic, which may provide a hydrophobic, porous plastic for air venting. In some embodiments, the membrane filter 61 may include polyvinylidene fluoride or another suitable material to provide reliable air venting. In some embodiments, the membrane filter 61 may include any suitable filter configured to pass air and not liquid known in the art.

Referring now to 2D, in some embodiments, as viewed from the top, the septum 38 may generally include an hourglass shape. In some embodiments, a distal end 60 of the septum 38 is generally bell shaped and configured to form the interface 50 with the inner surface 46 of the connector 26. In some embodiments, a proximal end 62 of the septum 38 may be similarly configured to form the interface 50 with the inner surface 46 of the connector 26. However, the proximal end 62 of the septum 38 further includes the docking portion 54 of the septum 38, as discussed in detail above. In some embodiments, a middle portion 64 of the septum 38 thins thereby forming an expansion void 66 on either side of the middle portion 64 between the septum 38 and the inner surface 46 of connector 26. In some embodiments, the middle portion 64 of the septum 38 is generally located directly under the flow control button 36. As such, in some embodiments, when the flow control button 36 is depressed, the middle portion 64 of the septum is actuated.

In some embodiments, the middle portion 64 of the septum 38 may include the slit 52. In some embodiments, the slit 52 is a physical opening through the septum 38 providing a pathway through the septum 38 whereby a fluid may move through the septum 38. In some embodiments, the septum 38 may be configured such that the slit 52 is biased in a closed position, as illustrated. In some embodiments, the slit 52 may be biased to an open position by depressing the flow control button 36 or by forcing a probe through the slit 52 via the docking portion 54 of the septum 38 after the vent plug 30 is removed. In some embodiments, as the slit 52 is biased to an open position, the middle portion 64 of the septum 38 divides into a first half 74 and second half 76, each half expanding radially outward into the provided expansion void 66.

Referring now to FIG. 2E, in some embodiments, the septum 38 may include a crease 68. In some embodiments, the crease 68 may include a linear score on an outer surface 70 of the septum 38 that runs generally parallel to the connector body 32. In some embodiments, the crease 68 may be positioned at the apex of the middle portion 64 so as to run horizontally along the middle portion 64. In some embodiments, the crease 68 may be characterized as a thinned region of the outer surface 70 of the septum 38. In some embodiments, the crease 68 may weaken the structural integrity of the middle portion 64 wall thereby increasing the flexibility of the thinned region of the septum 38. As such, the crease 68 may facilitate the middle portion 64 to bend towards the inner surface 46 of the connector 26 when the flow control button 36 is depressed. In this manner, a user may depress the flow control button 36 and actuate the septum 38 wherein the outer surface 70 of the septum 38 bends outwardly towards the inner surface 46 of the connector 26.

In some embodiments, the septum 38 may include an inner membrane 72. In some embodiments, the inner membrane 72 may include a first half 74 and a second half 76, each half being separated by the slit 52. In some embodiments, the inner membrane 72 may include the same material as the remainder of the septum 38 and may include any thickness necessary to prevent unwanted passage of a fluid through the septum 38. In some embodiments, the first and second halves 74, 76 of the inner membrane 72 may include a sealing surface 78, 80, respectively. In some embodiments, the inner membrane 72 may be configured such that the sealing surfaces 78, 80 of the first and second halves 74, 76 maintain contact with one another thereby forming a physical barrier preventing a fluid from passing through the septum 38. The sealing surfaces 78, 80 may be further modified to include complimentary surface designs. As such, the sealing surfaces 78, 80 may interlock or otherwise combine to further prevent the passage of fluid through the septum 38.

In some embodiments, as previously discussed, a user may depress the flow control button 36 to actuate the septum 38. In some embodiments, as the crease 68 of the septum 38 bends outwardly towards the inner surface 46 of the connector 26, the attached first and second halves 74, 76 of the inner membrane 72 may be likewise pulled outwardly towards the inner surface 46 of the connector 26. As such, the sealing surfaces 78, 80 of the inner membrane 72 may be drawn apart thereby opening the slit 52 of the septum 38 and enlarging the pathway through the septum 38.

In some embodiments, a portion of the material of the septum 38 may be removed to form a notch 82 near the underside of the flow control button 36. In some embodiments, the notch 82 may be provided to further ensure that the downward force of the depressed flow control button 36 is focused on the slit 52 of the septum 38. In this manner, the downward force of the depressed flow control button 36 may be evenly distributed on the slit 52 thereby equally biasing each crease 68 of the middle portion 64 to bend outwardly towards the inner surface 46 of the connector 26, as previously described.

Referring now to FIG. 2F, from the proximal end 56 of the connector 26, the first and second halves 74, 76 of the inner membrane 72 may be observed, according to some embodiments. In some embodiments, the proximal end 56 of the connector 26 may be modified to include a set of threads 84 or another feature for facilitating adapters or other components of an infusion system.

Referring now to FIG. 2G, in some embodiments, the inner membrane 72 may be positioned within the lumen of the septum 38 so as to divide the septum 38 into a first chamber 86 and a second chamber 88. In some embodiments, the first chamber 86 may be generally defined as the enclosed space spanning between the distal end 35 of the connector 26 and a distal side 92 of the inner membrane 72. In some embodiments, the second chamber 88 may be generally defined as the enclosed space spanning between a proximal side 93 of the inner membrane 72 and the threads 31. In some embodiments, when the inner membrane 72 is in a closed state, as illustrated, a fluid may be contained within the first chamber 86 while the second chamber 88 remains isolated from the fluid within the first chamber 86. Conversely, in some embodiments, a fluid may be contained within the second chamber 88 while the first chamber 86 remains isolated from the fluid within the second chamber 88. In this manner, a user may control the flow of a fluid through the septum 38 by controlling the open or close state of the inner membrane 72 of the septum 38.

Referring now to FIG. 2H, in some embodiments, the septum 38 may be opened by depressing the flow control button 36. In some embodiments, when the flow control button 36 is depressed, the middle section 42 may exert a downward 94 force on the middle portion 64 of the septum 38. In some embodiments, when the middle portion 64 is depressed in a downward 94 direction, the crease 68 of the middle portion 64 may bends outwardly towards the inner surface 46 of the connector 26, as shown in FIG. 2I. With continued reference to FIGS. 2H-2I, when the middle portion 64 bends outwardly into the expansion void 66, the first and second halves 74, 76 of the inner membrane 72 may be separated thereby exposing a pathway 96 through the inner membrane 72.

In some embodiments, the catch 44 may also be provided as part of the flow control button 36. In some embodiments, the catch 44 may be positioned such that when the flow control button 36 is depressed, the catch 44 passes through the window 34 and into the lumen 39 of the connector 26. In some embodiments, after the catch 44 is within the lumen 39, the catch 44 compatibly engages the inner surface 46 of the connector 26 thereby holding the flow control button 36 in a depressed position, as illustrated. In some embodiments, the catch 44 may be disengaged from the inner surface 46 of the connector 26 by simultaneously depressing the flow control button 36 and biasing the flow control button 36 in the forward direction 48, thereafter releasing the flow control button 36 to a relaxed position.

Referring now to FIG. 2J, in some embodiments, when the flow control button 36 is depressed, the middle section 42 of the flow control button 36 may exert a downward 94 force on the middle portion 64 of the septum 38. In some embodiments, the crease 68 of the middle portion 64 may bend outwardly towards the inner surface 46 of the connector 26. As such, the first and second halves 74, 76 of the inner membrane 72 may be separated to expose the pathway 96 through the inner membrane 72.

Referring now to FIG. 2K, in some embodiments, from the proximal end 56 of the connector 26, the first and second halves 74, 76 of the inner membrane 72 may be observed. In some embodiments, once actuated by depressing the flow control button 36, the first and second halves 74, 76 are separated revealing a pathway 96 through the septum 38. In some embodiments, by depressing the flow control button 36, the first and second halves 74, 76 of the septum 38 may be biased outwardly and separated in a manner as illustrated.

Referring now to FIG. 2L, in some embodiments, the first and second halves 74, 76 (see second half 76 in FIG. 2E, for example) of the inner membrane 72 of the septum 38 may be biased outwardly towards the inner surface 46 as the flow control button 36 is depressed. Specifically, a middle section 42 of the flow control button 36 may be provided to exert a focused, downward 94 force on inner membrane 72 of the septum 38. In some embodiments, the downward 94 force causes the first and second halves 74, 76 of the membrane to relax outwardly and separate from one another revealing the pathway 96 through the septum 38.

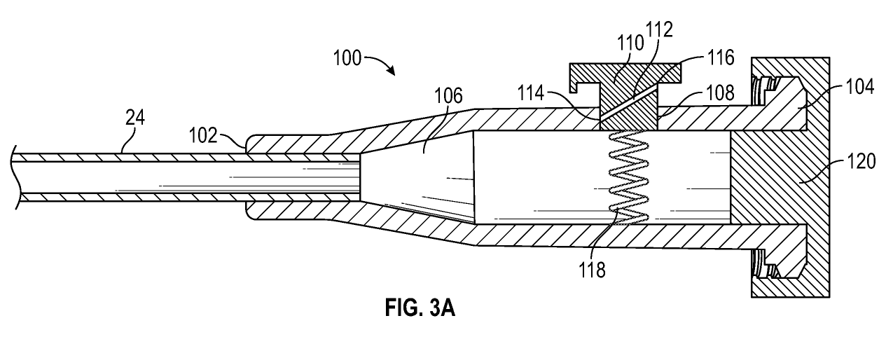

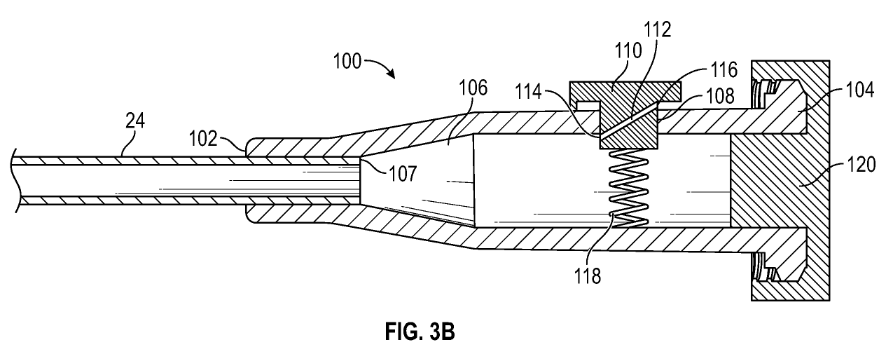

Referring now to FIGS. 3A-3B, in some embodiments, a connector 100 of a catheter system may include a distal end 102, a proximal end 104, a lumen 106 extending through the distal end 102 of the connector 100 and the proximal end 104 of the connector 100. In some embodiments, the connector 100 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation. In some embodiments, a proximal end 107 of the extension tubing 24 may be integrated with the connector 100, such as in an interference fit. In some embodiments, the connector 100 may include a slot 108 disposed between the distal end 102 of the connector 100 and the proximal end 104 of the connector 100. In some embodiments, the lumen 106 may be in fluid communication with the extension tubing 24.

In some embodiments, a button 110 may be disposed within the slot 108. In some embodiments, the button 110 may include a venting pathway 112 extending through the button 110. In some embodiments, the venting pathway 112 may include a first end 114 and a second end 116 laterally offset from the first end 114. In some embodiments, the venting pathway 112 may include a membrane filter to pass air and not blood and/or may be sized and configured to pass air and not blood. Thus, the venting pathway 112 may limit blood exposure. In embodiments, when the venting pathway 112 is sized and configured to pass air and not blood, surface tension due to cohesive forces between liquid molecules may prevent liquid from passing through the venting pathway 112, while air may be allowed to pass.

In some embodiments, the membrane filter may include a hydrophobic membrane, such as, for example, polytetrafluoroethylene (PTFE) to provide hydrophobic properties and chemical resistance and facilitate airflow. In some embodiments, the membrane filter may include polyethylene, polypropylene, or another plastic, which may provide a hydrophobic, porous plastic for air venting. In some embodiments, the membrane filter may include polyvinylidene fluoride or another suitable material to provide reliable air venting. In some embodiments, the membrane filter may include any suitable filter configured to pass air and not liquid known in the art.

In some embodiments, when the button 110 is in a raised position, as illustrated, for example, in FIG. 3A, the first end 114 may be blocked from fluid communication with the lumen 106, and the second end 116 may be in fluid communication with ambient air. In these embodiments, air may not be able to escape through the slot 108, which may be sealed with respect to the button 110. In some embodiments, when the button 110 is depressed, as illustrated, for example, in FIG. 3B, the first end 114 may be disposed within the lumen 106, and the second end 116 may be in fluid communication with the ambient air such that air vents through the venting pathway 112 from the lumen 106 into the ambient air.

In some embodiments, the connector 100 may include a spring 118 disposed within the lumen 106 and configured to return the button 110 from the depressed position to the raised position. In some embodiments, a cap 120 may be coupled to the proximal end 104 of the connector to seal the proximal end 104 of the connector 100 and prevent any blood leakage through the proximal end 104. In other embodiments, a needleless access connector or other type of flow controller may be coupled to the proximal end 104 of the connector 100.

In some embodiments, the button 110 may act as a bleed off valve that allows air to exit the catheter system through the venting pathway 112 within the button 110 when the button 110 is depressed, as opposed to allowing air to exit the catheter system through the proximal end 104 of the connector 100 as in FIG. 2, for example. In some embodiments, the button 110 may be in the raised position during insertion of the catheter system into the artery such that pulsatile blood flow is visible within the extension tubing 24. In some embodiments, after the pulsatile blood flow is observed by the clinician in the extension tubing 24 indicating the catheter 14 is inserted into the artery, the clinician can depress the button 110 to allow venting of air through the venting pathway 112 and blood to travel through the extension tubing 24 and to the connector 100 prior to attachment of the IV line or another device to the connector 26. In some embodiments, after blood is sufficiently advanced, the clinician can release pressure on the button 110 to block the venting pathway 112, so that the clinician can then remove the cap 120 and attach the IV line or other device to the proximal end 104 of the connector 100.

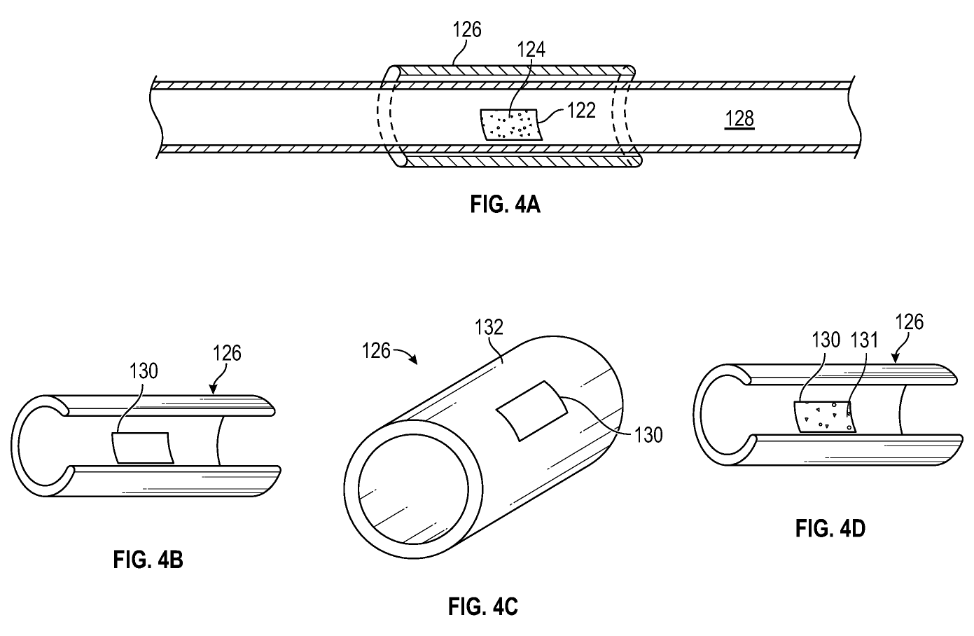

Referring now to FIGS. 4A-4D, the extension tube 24 of a catheter system may include a window 122 disposed between the distal end of the extension tubing 24 and the proximal end of the extension tubing 24. In some embodiments, the catheter system may be similar or identical to the catheter system 2 of FIG. 1 and/or the catheter system 10 of FIG. 2 in terms of one or more components and/or operation. In some embodiments, the window 122 may include a membrane filter 124 therein. In some embodiments, the membrane filter 124 may be configured to pass air and not blood thereby preventing blood from exiting the window 122.

In some embodiments, a particular connector may be coupled to the proximal end of the extension tubing 24. In some embodiments, the particular connector may include a distal end, a proximal end, a lumen extending through the distal end of the particular connector and the proximal end of the particular connector. In some embodiments, the lumen of the particular connector may be in fluid communication with the extension tubing 24.

In some embodiments, a cover 126 may be configured to cover the window 122 to block fluid communication between a lumen 128 of the extension tubing 24 and the ambient air. In some embodiments, the cover 126 may be configured to be rotated or slid with respect to the extension tubing 24 to uncover the window 122 and allow the air to pass through membrane filter 124 from the lumen 128 of the extension tubing 24 to the ambient air. In some embodiments, providing an air venting location within the extension tube 24 or near a middle of the extension tubing 24 allows pressurization in the extension tube 24 to begin at the air venting location of the extension tubing 24 instead of at a distal tip of the catheter, thus moving visualization of the pulsatile blood flow proximally within the catheter system.

As illustrated, for example, in FIGS. 4A-4B, in some embodiments, the cover 126 may include a snap-on cover extending partially around a circumference of the extension tubing 24 and having another window 130. In some embodiments, the other window may include an open space without a membrane filter therein due to the window 122 providing blood control via the membrane filter 124. In some embodiments, when the snap-on cover is in a first position, the window 122 and the other window 130 may be misaligned such that the cover 126 prevents the air from venting through the membrane filter 124 into ambient air. In some embodiments, the snap-on cover may be configured to slide in a distal or proximal direction along the extension tubing 24.

In some embodiments, the snap-on cover may be configured to rotate around a central axis of the extension tubing 24 to a second position. In some embodiments, when the snap-on cover is in the second position, the window 122 and other window 130 may be aligned or overlapping such that air is configured to vent through the membrane filter 124 into the ambient air.

As illustrated, for example, in FIG. 4C, in some embodiments, the cover 126 may include an annular body 132 extending around an entirety of a circumference of the extension tubing 24 and the other window 130. In some embodiments, when the annular body 132 is in a first position, the window 122 and the other window 130 may be misaligned or non-overlapping such that the cover 126 prevents the air from venting through the membrane filter into ambient air. In some embodiments, the annular body 132 may be configured to rotate around the extension tubing 24 to a second position. For example the annular body 132 may rotate around the central axis of the extension tube 24 between the first position and the second position. In some embodiments, when the annular body 132 is in the second position, the window 122 and other window 130 may be aligned such that air is configured to vent through the membrane filter 124 into the ambient air.

In some embodiments, if arterial blood pressure is very low so that pulsatile blood flow does not compress gas enough to be present in the extension tubing 24, the window 122 and the cover 126 may be aligned to allow free flow of blood within the extension tubing 24 until the blood reaches a desired point of visualization. After the blood reaches the desired point of visualization, which may be between the window 122 and the catheter adapter, the window 122 and cover 126 can be misaligned to allow visualization of the pulsatile blood flow within the extension tubing 24 and ensure continuous feedback regarding insertion success.

As illustrated, for example, in FIG. 4D, in some embodiments, the other window 130 may include a membrane filter 131 therein. In some embodiments, the membrane filter 131 may be permeable to air and not blood. In these embodiments, the cover 126 may be fixed with respect to the extension tube 24 such as via adhesive or another means. In some embodiments, the cover 126 may be fixed with respect to the extension tube 24 such that the other window 130 is permanently aligned with the window 122. In these embodiments, the window 122 permanently aligned with the other window 130 may allow venting of air and blood to travel through the extension tubing 24 until the blood reaches the window 122.

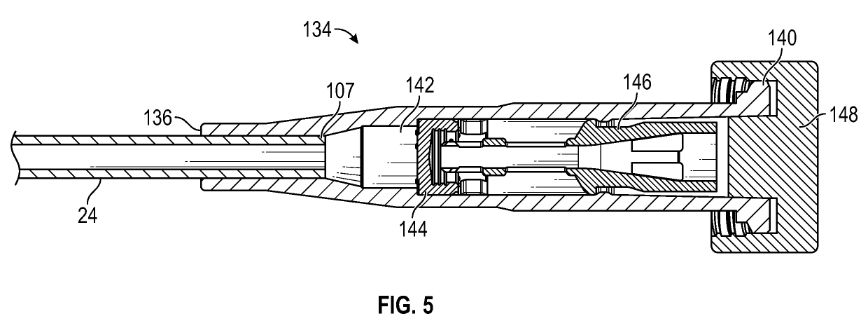

Referring now to FIG. 5, in some embodiments, a connector 134 of a catheter system may be coupled to the proximal end 107 of the extension tubing 24. In some embodiments, the connector 134 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the connector 134 may include a distal end 136, a proximal end 140, and a lumen 142 extending through the distal end 136 of the connector 134 and the proximal end 140 of the connector 134. In some embodiments, the lumen 142 may be in fluid communication with the extension tubing 24. In some embodiments, a septum 144 and a septum actuator 146 may be disposed within the lumen 142.

In some embodiments, a cap 120 may be coupled to the proximal end 104 of the connector 134 to seal the proximal end 104 of the connector 100. In some embodiments, the cap 120 may not include a male luer configured to contact the septum actuator 146 and move the septum actuator 146 distally. Thus, the septum 144 may be closed. In some embodiments, the cap 120 without the male luer configured to contact and move the septum actuator 146 may seal the proximal end 140 to allow visualization of the pulsatile blood flow within the extension tube 24 when the catheter system is inserted into the artery. In some embodiments, when the cap 120 is removed, blood from the artery may prime the extension tubing 24 to the septum 144. In some embodiments, after the cap 120 is removed, the IV line or other device having a male luer may be coupled to the proximal end 140 of the connector 134, opening the septum 144 by pushing the septum actuator 146 distally. Thus, the connector 134 may facilitate a bloodless attachment of the IV line or other device.

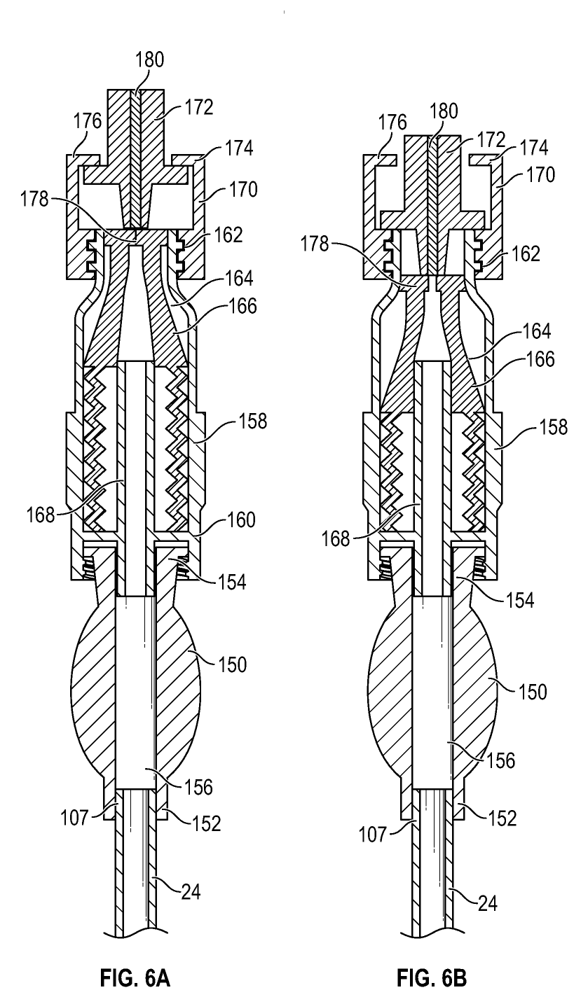

Referring now to FIGS. 6A-6B, in some embodiments, a connector 150 of a catheter system may be coupled to the proximal end 107 of the extension tubing 24. In some embodiments, the connector 150 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the connector 150 may include a distal end 152, a proximal end 154, and a lumen 156 extending through the distal end 152 of the connector 150 and the proximal end 154 of the connector 150. In some embodiments, the lumen 156 may be in fluid communication with the extension tubing 24.

In some embodiments, another connector 158 may be coupled to the proximal end 154 of the connector 150. In some embodiments, the other connector 158 may include a distal end 160, a proximal end 162, and a lumen 164 extending through the distal end 160 and the proximal end 162. In some embodiments, the other connector 158 may correspond to a MAXZERO™ Needle-free connector, available from Becton, Dickinson & Company of Franklin Lakes, New Jersey, or another suitable connector. In some embodiments, the other connector 158 may include a septum 166 and/or a septum actuator 168, which may be fixed.

In some embodiments, a housing 170 may be coupled to the proximal end 162 of the other connector 158. In some embodiments, the housing 170 may be threadedly coupled to the proximal end 162 of the other connector 158, which may aid removal of the housing 170 so an infusion or blood draw device can be coupled to the proximal end 162 of the other connector 158. In some embodiments, a vent plug 172 may be movable within the housing 170 from a first position, illustrated, for example, in FIG. 6A, and a second position, illustrated, for example, in FIG. 6B. In some embodiments, a proximal end 174 of the housing 170 may include a flange 176 configured to act as a stop when the vent plug 172 is in the first position to prevent proximal movement of the vent plug 172. In some embodiments, the flange 176 may be annular.

In some embodiments, the septum 166 may include a slit 178 that is biased in a closed position to prevent fluid flow through the lumen 164. In some embodiments, in response to the vent plug 172 being moved from the first position to the second position, the vent plug 172 may open the slit 178 of the septum 166 such that a venting pathway 180 is in fluid communication with the lumen 156 of the connector 150 and air vents through the venting pathway 180 from the lumen 156 into the ambient air. In some embodiments, when the vent plug 172 is in the second position, blood may flow through the catheter system and into the extension tubing 24, priming the catheter system with blood.

In some embodiments, the venting pathway 180 may extend through the vent plug 172 generally parallel to a central axis of the vent plug 172. In some embodiments, the venting pathway 180 may be sized and configured to pass air and not blood. In some embodiments, a membrane filter configured to pass air and not blood may be disposed within the venting pathway 180. In some embodiments, an entirety of the vent plug 172 may be constructed of the membrane filter, which, as explained above, may include a hydrophobic membrane or another suitable material to provide air venting.

In some embodiments, the vent plug 172 may act as a button to allow a manual pushing action from the first position to the second position. In some embodiments, after release of pressure on the vent plug 172, the septum 166 may be resilient and act as a spring that returns the vent plug 172 from the second position to the first position, closing the septum 166 without blood leakage.

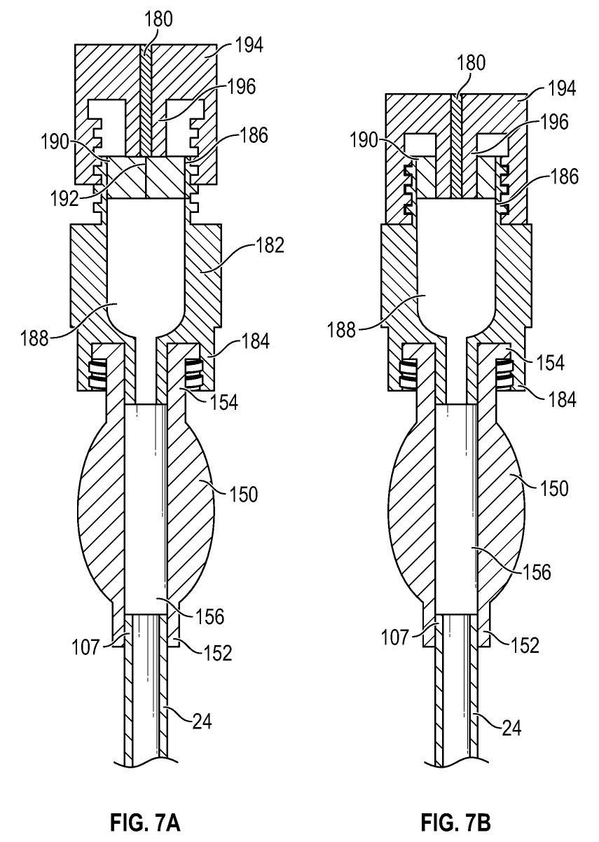

Referring now to FIGS. 7A-7B, in some embodiments, the connector 150 of a catheter system may be coupled to the proximal end 107 of the extension tubing 24. In some embodiments, the connector 150 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the connector 150 may include the distal end 152, the proximal end 154, and the lumen 156 extending through the distal end 152 of the connector 150 and the proximal end 154 of the connector 150. In some embodiments, the lumen 156 may be in fluid communication with the extension tubing 24.

In some embodiments, another connector 182 may be coupled to the proximal end 154 of the connector 150. In some embodiments, the other connector 182 may include a distal end 184, a proximal end 186, and a lumen 188 extending through the distal end 184 and the proximal end 154. In some embodiments, the other connector 182 may correspond to a BD Q-SYTE™ Needle-less connector, available from Becton, Dickinson & Company of Franklin Lakes, New Jersey, or another suitable connector. In some embodiments, the other connector 182 may reduce a bulkiness and weight of the catheter system, as well as reduce dead space. In some embodiments, the other connector 182 may include a septum 190, which may include a slit 192.

In some embodiments, when coupled to the catheter system, a vent plug 194 may be movable between a first position, illustrated, for example, in FIG. 7A, and a second position, illustrated, for example, in FIG. 7B. In some embodiments, the vent plug 194 may include a venting pathway 180 extending through the vent plug 194. In some embodiments, the venting pathway 180 may be sized and configured to pass air and not blood. In some embodiments, a membrane filter may be disposed within the venting pathway 180.

In some embodiments, in response to the vent plug 194 being moved from the first position, illustrated, for example, in FIG. 7A, to the second position, illustrated, for example, in FIG. 7B, the venting pathway 180 may be in fluid communication with the lumen 156 of the connector 150 and/or the lumen 188 of the other connector 182 such that air vents through the venting pathway 180 from the lumen 156 and/or the lumen 188 into the ambient air. In some embodiments, when the vent plug 194 is in the first position, the pulsatile blood flow may be visible within the extension tube 24. In some embodiments, when the vent plug 194 is in the second position, blood may flow through the catheter system and into the extension tubing 24, priming the catheter system with blood.

In some embodiments, the vent plug 194 may be threaded with respect to the other connector 182 from the first position and the second position. In some embodiments, the slit 192 of the septum 190 may be biased in a closed position to prevent fluid flow through the lumen. In some embodiments, in response to the vent plug 194 being threaded with respect to the other connector 182 from the first position to the second position, an actuator 196 of the vent plug 194 may open the slit 192 of the septum 190 such that the venting pathway 180 is in fluid communication with the lumen 156 of the connector 150 and air vents through the venting pathway 180 from the lumen 156 into the ambient air.

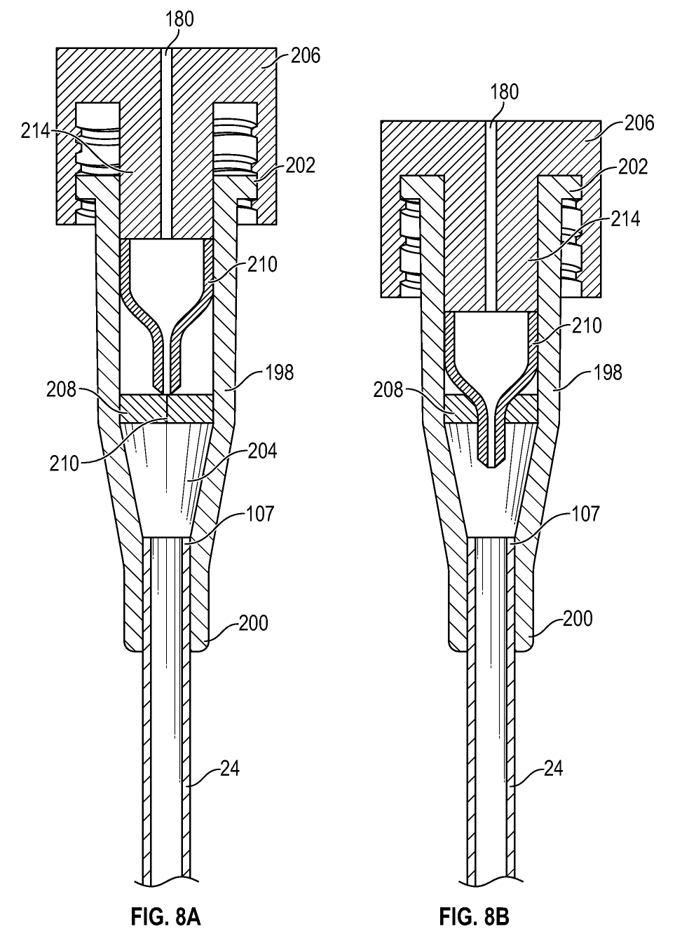

Referring now to FIGS. 8A-8B, in some embodiments, a connector 198 of a catheter system may be coupled to the proximal end 107 of the extension tubing 24. In some embodiments, the connector 198 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the connector 198 may include a distal end 200, a proximal end 202, and a lumen 204 extending through the distal end 200 of the connector 198 and the proximal end 202 of the connector 198. In some embodiments, the lumen 204 may be in fluid communication with the extension tubing 24.

In some embodiments, a vent plug 206 may be coupled to the proximal end 202 of the connector 198. In some embodiments, the catheter system may include a septum 208 disposed within the lumen 204 and a septum actuator 210 disposed within the lumen 204. In some embodiments, the vent plug 206 may be threaded with respect to the connector 198 from a first position, illustrated, for example, in FIG. 8A, and a second position, illustrated, for example, in FIG. 8B. In some embodiments, in response to the vent plug 206 being threaded with respect to the connector 198 from the first position to the second position, the vent plug 206 may push the septum actuator 210 distally to open the septum 208 such that a venting pathway 180 of the vent plug 206 is in fluid communication with the lumen 204 and air vents through the venting pathway 180 from the lumen 204 into the ambient air.

In some embodiments, the septum 208 may include a slit 212 that may be penetrated by an actuator 214 of the septum actuator 210 when the septum actuator 210 is pushed distally by the vent plug 206. In some embodiments, the actuator 214 may include a male luer. In some embodiments, the venting pathway 180 may be sized and configured to pass air and not blood. In some embodiments, a membrane filter may be disposed within the venting pathway 180.

In some embodiments, when the vent plug 206 is in the first position, the pulsatile blood flow may be visible within the extension tube 24. In some embodiments, when the vent plug 206 is in the second position, blood may flow through the catheter system and into the extension tubing 24, priming the catheter system with blood. In some embodiments, the vent plug 206 may be unthreaded from the second position to the first position, which may cause the septum actuator 210, which may be spring-based, to move proximally and remove from the septum 208, closing the septum 208, and allowing for safe connection of the IV line or other device to the proximal end 202 without exposure to blood.

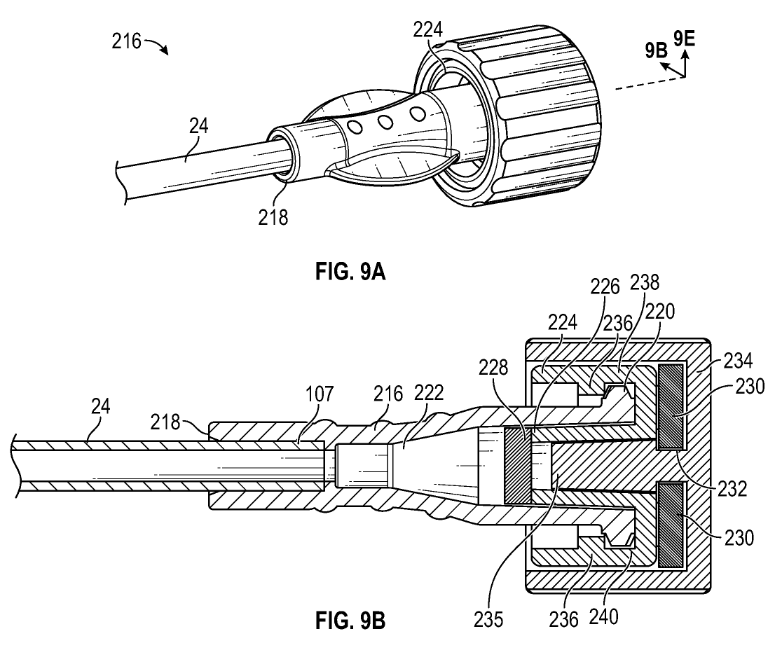

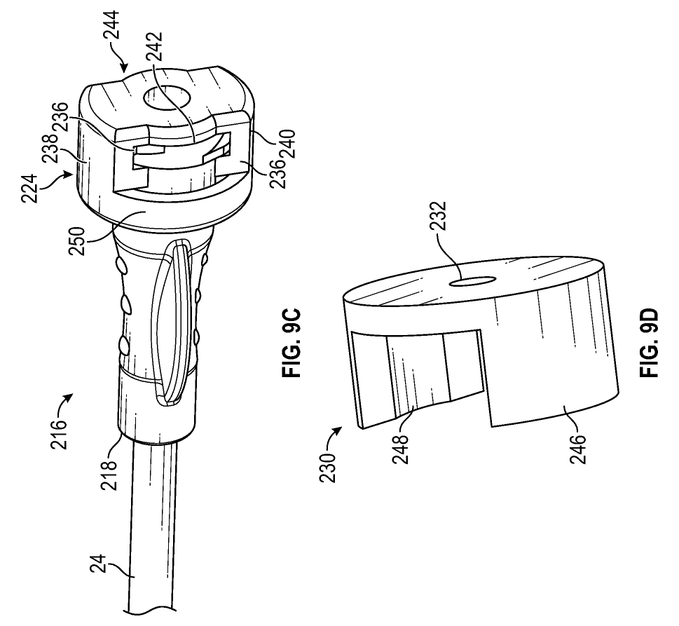

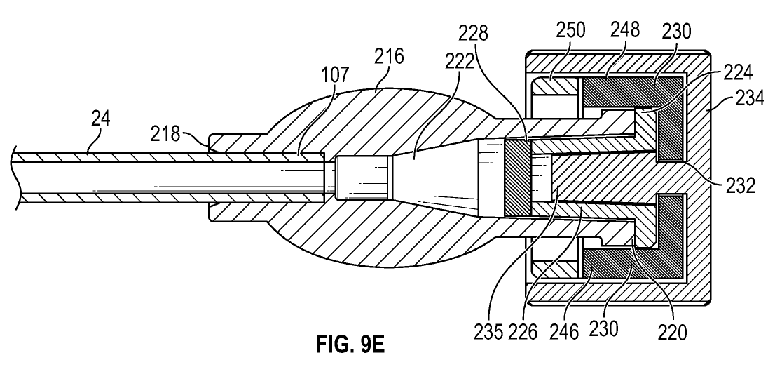

Referring now to FIGS. 9A-9E, a connector 216 of a catheter system may be coupled to the proximal end 107 of the extension tubing 24. In some embodiments, the connector 216 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the connector 216 may include a distal end 218, a proximal end 220, and a lumen 222 extending through the distal end 218 of the connector 216 and the proximal end 220 of the connector 216. In some embodiments, the lumen 222 may be in fluid communication with the extension tubing 24.

In some embodiments, the catheter system may include a first cap 224 threadedly coupled to the proximal end 220 of the connector 216. In some embodiments, the first cap 224 may include a male luer 226 disposed within the proximal end 220 of the connector 216 and a venting pathway 180 extending through the male luer 226. In some embodiments, the venting pathway 180 may be configured to pass air and not blood. In some embodiments, the venting pathway 180 may include a membrane filter 228 disposed at a distal end of the venting pathway 180.

In some embodiments, the catheter system may include a lock piece 230 having an aperture 232 therethrough. In some embodiments, the lock piece 230 may prevent the first cap 224 from being unthreaded from the proximal end 220 of the connector 216. In some embodiments, the catheter system may include a second cap 234 extending through the aperture 232 and having an extension 235 forming a seal inside the male luer 226. In some embodiments, the lock piece 230 may be configured to be removed from the catheter system with the second cap 234 before the first cap 224 can be unthreaded from the proximal end 220 of the connector 216.

In some embodiments, the first cap 224 may include threading 236 that is segmented and disposed on opposing portions 238, 240 of the first cap 224 spaced apart by a first gap 242 and second gap 244. In some embodiments, the lock piece 230 may include a first arm 246 disposed within the first gap 242 and a second arm 248 disposed within the second gap 244 thereby preventing the first cap 224 from rotating and being unthreaded from the proximal end 220 of the connector 216. In some embodiments, because the first cap 224 cannot be removed from the connector 216 without first removing the second cap 234 and the lock piece 230, the first cap 224 and the second cap 234 ensure a proper order of removal by the clinician.

In some embodiments, when both the first cap 224 and the second cap 234 are coupled to the catheter system as illustrated in FIG. 9B, the pulsatile blood flow may be visible within the extension tube 24 because the second cap 234 may block the venting pathway 180. In some embodiments, when the second cap 234 is removed, blood may flow through the catheter system and into the extension tubing 24, priming the catheter system with blood. In some embodiments, the first cap 224 may be removed after the second cap 234 is removed to allow connection of the IV line or other device to the proximal end 220 of the connector 216.

In some embodiments, the first cap 224 may include an annular ring 250 proximate the first gap 242 and the second gap 244. In some embodiments, the annular ring 250 may provide support to the first cap 224 and/or facilitate twisting to unthread the first cap 224 with respect to the connector 216 when it is desired to remove the first cap 224 and connect the IV line or other device to the proximal end 220 of the connector 216.

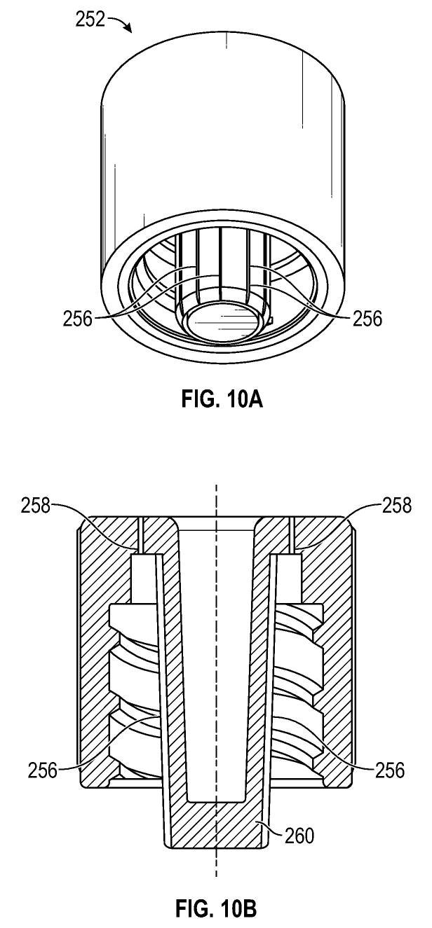

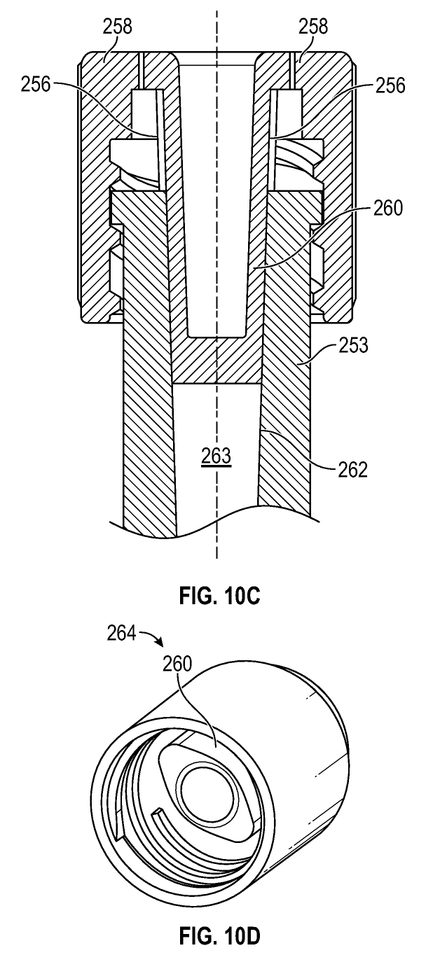

Referring now to FIGS. 10A-10C, in some embodiments, a vent plug 252 of a catheter system may be coupled to a proximal end of a connector 253. In some embodiments, the connector 253 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the vent plug 252 may be movable between a first position and a second position when coupled to the connector 253. The first position may be illustrated in FIG. 10C, according to some embodiments. In some embodiments, the vent plug 252 may include a venting pathway extending through the vent plug 194. In some embodiments, the venting pathway may include one or more grooves 256 and one or more apertures 258.

In some embodiments, the apertures 258 may extend through a base of the vent plug 252. In some embodiments, the apertures 258 may be sized and configured to pass air and not blood. In some embodiments, the apertures 258 may include a membrane filter to pass air and not blood.

In some embodiments, the vent plug 252 may be unthreaded in a proximal direction with respect to the connector 253 from the first position and the second position. In some embodiments, the vent plug 252 may include a male luer 260 having the grooves 256. In some embodiments, the grooves 256 may be spaced around an exterior surface of the male luer 260. In some embodiments, when the vent plug 252 is in the second position, the grooves 256 may be uncompressed and/or sized and configured to pass air and not blood, similar to FIG. 10B.

In some embodiments, when the vent plug 252 is in the first position or in response to the vent plug 252 being threaded with respect to the connector 253 from the second position to the first position, the grooves 256 may be compressed and/or pressure within the grooves 256 increases such that the venting pathway is blocked. In some embodiments, a taper of the internal wall 262 forming a lumen 263 may be configured to compress the grooves 256 when the vent plug 252 is in the first position and not the second position to prevent air from flowing through the grooves 256. Thus, a seal may be formed between the male luer 260 and the connector 253.

In some embodiments, in response to the vent plug 252 being moved from the first position to the second position, the grooves 256 may be uncompressed such that the venting pathway is in fluid communication with a lumen of the connector 253 and air vents through the venting pathway from the lumen of the connector 253. In some embodiments, the vent plug 252 may be constructed of plastic, silicone, or another suitable material configured to allow the grooves 256 to compress such that air does not pass through.

Referring now to FIGS. 10D, in some embodiments, a vent plug 264 of a catheter system may be coupled to a proximal end of the connector 150 (see, for example FIGS. 6-7) or another suitable connector. In some embodiments, a particular connector coupled to the vent plug 264 may replace the connector 9 in the catheter system 2 of FIG. 1 or the catheter system 10 of FIG. 2. In some embodiments, the catheter system may be similar or identical to the catheter system 2 of FIG. 1 or the catheter system 10 of FIG. 2 in terms of one or more components and/or operation.

In some embodiments, the vent plug 264 may be unthreaded in a proximal direction with respect to the connector 150 from the first position and the second position, similar to the vent plug 252 of FIGS. 10A-10C. In some embodiments, a portion of the vent plug 264 configured to insert into the lumen 263 of the vent plug 264 may be oblong or oval. Thus, in some embodiments, when the vent plug 264 is in the second position, air may be configured to flow between the portion of the vent plug 264 and an internal wall of the connector 150. In some embodiments, the internal wall of the connector 150 may form a lumen have a tapered shape such that a proximal portion of the lumen has a smaller diameter than a distal portion of the lumen. In some embodiments, when the vent plug 264 is in the first position or in response to the vent plug 264 being moved from the second position to the first position, the portion may be configured to compress and seal the lumen 263. In some embodiments, the internal wall may be configured to compress the portion of the vent plug 264 that is oblong or oval when the vent plug 252 is in the first position to form a seal between the portion of the vent plug 264 and the connector 150.

In some embodiments, the apertures 258 may extend through a base of the vent plug 264 to allow venting of air therethrough. In some embodiments, the portion of vent plug 264 may be constructed of plastic, silicone, or another suitable material configured to compress to form a seal as described.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

1. A catheter system, comprising:
a catheter adapter, comprising a distal end, a proximal end, and a side port in between the distal end of the catheter adapter and the proximal end of the catheter adapter;
a catheter extending distally from the distal end of the catheter adapter;
an extension tubing comprising a distal end and a proximal end, wherein the distal end of the extension tubing is coupled to the side port;
a connector coupled to the proximal end of the extension tubing, wherein the connector comprises a distal end, a proximal end, a lumen extending through the distal end of the connector and the proximal end of the connector, and a window disposed between the distal end of the connector and the proximal end of the connector, wherein the lumen is in fluid communication with the extension tubing;
a septum positioned within the lumen, wherein the septum comprises a slit that is biased in a closed position to prevent fluid flow through the lumen;
a button disposed within the window, wherein when the button is pressed, the button causes the slit of the septum to open thereby creating a fluid pathway through the lumen;
a vent plug coupled to the proximal end of the connector, wherein the vent plug is configured to vent air from the extension tubing and through the septum when the button causes the slit of the septum to open.

2. The catheter system of claim 1, wherein the vent plug comprises a venting pathway extending through the vent plug, wherein the vent plug further comprises a membrane filter disposed within the venting pathway and configured to pass air but not blood.

3. A catheter system, comprising:
a catheter adapter, comprising a distal end, a proximal end, and a side port in between the distal end of the catheter adapter and the proximal end of the catheter adapter;
a catheter extending distally from the distal end of the catheter adapter;
an extension tubing comprising a distal end and a proximal end, wherein the distal end of the extension tubing is coupled to the side port;
a connector coupled to the proximal end of the extension tubing, wherein the connector comprises a distal end, a proximal end, a lumen extending through the distal end of the connector and the proximal end of the connector, and a slot disposed between the distal end of the connector and the proximal end of the connector, wherein the lumen is in fluid communication with the extension tubing; and
a button disposed within the slot, wherein the button comprises a venting pathway extending through the button, wherein the venting pathway comprises a first end and a second end laterally offset from the first end, wherein the venting pathway is configured to pass air and not blood, wherein when the button is in a raised position, the first end is blocked from fluid communication with the lumen and the second end is in fluid communication with ambient air, wherein when the button is depressed, the first end is disposed within the lumen and the second end is in fluid communication with ambient air such that air vents through the venting pathway from the lumen into the ambient air.

4. The catheter system of claim 3, wherein the connector further comprises a spring disposed within the lumen and configured to return the button from the depressed position to the raised position.

5. The catheter system of claim 3, further comprising a cap sealing the proximal end of the connector.

6. A catheter system, comprising:
a catheter adapter, comprising a distal end, a proximal end, and a side port in between the distal end of the catheter adapter and the proximal end of the catheter adapter;
a catheter extending distally from the distal end of the catheter adapter;
an extension tubing comprising a distal end, a proximal end, and a window disposed between the distal end of the extension tubing and the proximal end of the extension tubing, wherein the distal end of the extension tubing is coupled to the side port, wherein the window comprises a membrane filter therein, wherein the membrane filter is configured to pass air and not blood thereby preventing blood from exiting the window;
a connector coupled to the proximal end of the extension tubing, wherein the connector comprises a distal end, a proximal end, a lumen extending through the distal end of the connector and the proximal end of the connector, wherein the lumen is in fluid communication with the extension tubing;
a cover configured to cover the window to block fluid communication between a lumen of the extension tubing and the ambient air, wherein the cover is configured to be rotated or slid with respect to the extension tubing to uncover the window and allow the air to pass through membrane filter from the lumen of the extension tubing to the ambient air.

7. The catheter system of claim 6, wherein the cover comprises a snap-on cover extending partially around a circumference of the extension tubing and having another window, wherein when the snap-on cover is in a first position, the window and the other window are misaligned such that the cover prevents the air from venting through the membrane filter into ambient air, wherein the snap-on cover is configured to rotate around the extension tubing to a second position, wherein when the snap-on cover is in the second position, the window and other window are aligned such that air is configured to vent through the membrane filter into the ambient air.

8. The catheter system of claim 6, wherein the cover comprises an annular body extending around an entirety of a circumference of the extension tubing and having another window, wherein when the annular body is in a first position, the window and the other window are misaligned such that the cover prevents the air from venting through the membrane filter into ambient air, wherein the annular body is configured to rotate around the extension tubing to a second position, wherein when the annular body is in the second position, the window and other window are aligned such that air is configured to vent through the membrane filter into the ambient air.

9. A catheter system, comprising:
a catheter adapter, comprising a distal end, a proximal end, and a side port in between the distal end of the catheter adapter and the proximal end of the catheter adapter;
a catheter extending distally from the distal end of the catheter adapter;
an extension tubing comprising a distal end and a proximal end, wherein the distal end of the extension tubing is coupled to the side port;
a connector coupled to the proximal end of the extension tubing, wherein the connector comprises a distal end, a proximal end, and a lumen extending through the distal end of the connector and the proximal end of the connector, wherein the lumen is in fluid communication with the extension tubing; and
a first cap threadedly coupled to the proximal end of the connector, wherein the first cap comprises a male luer disposed within the proximal end of the connector and a venting pathway extending through the male luer, wherein the venting pathway is configured to pass air and not blood;
a lock piece comprising an aperture therethrough, wherein the lock piece prevents the first cap from being unthreaded from the proximal end of the connector;
a second cap extending through the aperture and forming a seal inside the male luer, wherein the lock piece is configured to be removed from the catheter system with the second cap before the first cap can be unthreaded from the proximal end of the connector.

10. The catheter system of claim 9, wherein the first cap comprises threading that is segmented and disposed on opposing portions of the first cap spaced apart by a first gap and second gap, wherein the lock piece comprises a first arm disposed within the first gap and a second arm disposed within the second gap thereby preventing the first cap from rotating and being unthreaded from the proximal end of the connector.

11. A catheter system, comprising:
a catheter adapter, comprising a distal end, a proximal end, and a side port in between the distal end of the catheter adapter and the proximal end of the catheter adapter;
a catheter extending distally from the distal end of the catheter adapter;
an extension tubing comprising a distal end and a proximal end, wherein the distal end of the extension tubing is coupled to the side port;
a connector coupled to the proximal end of the extension tubing, wherein the connector comprises a distal end, a proximal end, and a lumen extending through the distal end of the connector and the proximal end of the connector, wherein the lumen is in fluid communication with the extension tubing;
a vent plug coupled to the catheter system and movable between a first position and a second position when coupled to the catheter system, wherein the vent plug comprises a venting pathway extending through the vent plug, wherein the venting pathway is configured to pass air and not blood, wherein in response to the vent plug being moved from the first position to the second position, the venting pathway is in fluid communication with the lumen such that air vents through the venting pathway from the lumen into the ambient air.

12. The catheter system of claim 11, further comprising another connector coupled to the proximal end of the connector and having another lumen, wherein the catheter system further comprises a housing coupled to the proximal end of the connector, wherein the vent plug is movable within the housing from the first position and the second position, wherein the catheter system further comprises a septum positioned within the other connector, wherein the septum comprises a slit that is biased in a closed position to prevent fluid flow through the other lumen, wherein in response to the vent plug being moved from the first position to the second position, the vent plug opens the slit of the septum such that the venting pathway is in fluid communication with the lumen of the connector and air vents through the venting pathway from the lumen of the connector into the ambient air.

13. The catheter system of claim 11, wherein the catheter system further comprises another connector coupled to the proximal end of the connector and having another lumen, wherein the catheter system further comprises a septum disposed within the other connector, wherein the vent plug is threaded with respect to the other connector from the first position and the second position, wherein the septum comprises a slit that is biased in a closed position to prevent fluid flow through the other lumen, wherein in response to the vent plug being threaded with respect to the other connector from the first position to the second position, the vent plug opens the slit of the septum such that the venting pathway is in fluid communication with the lumen of the connector and air vents through the venting pathway from the lumen of the connector into the ambient air.

14. The catheter system of claim 11, wherein the vent plug is coupled to the proximal end of the connector, further comprising a septum disposed within the lumen and a septum actuator disposed within the lumen, wherein the vent plug is threaded with respect to the connector from the first position and the second position, wherein in response to the vent plug being threaded with respect to the connector from the first position to the second position, the vent plug pushes the septum actuator distally to open the septum such that the venting pathway is in fluid communication with the lumen and air vents through the venting pathway from the lumen into the ambient air.

15. The catheter system of claim 11, wherein the vent plug is coupled to the proximal end of the connector, wherein the vent plug is unthreaded with respect to the connector from the first position and the second position, wherein a portion of the vent plug configured to insert into the lumen is oblong, wherein in response to the vent plug being moved from the second position to the first position, the portion is configured to compress and seal the lumen.

16. The catheter system of claim 11, wherein the vent plug is coupled to the proximal end of the connector, wherein the vent plug is unthreaded with respect to the connector from the first position and the second position, wherein the vent plug comprises a male luer having a plurality of grooves, wherein in response to the vent plug being threaded with respect to the connector from the second position to the first position, the plurality of grooves are compressed or pressure within the plurality of grooves increases such that the venting pathway is blocked.

17. The catheter system of claim 1, wherein the lumen comprises an actuator stop that limits proximal movement of the actuator.

18. The catheter system of claim 1, wherein the button comprises a portion of the septum.

19. The catheter system of claim 1, wherein the button includes a wedged extension that catches on an edge of the window when the button is compressed thereby retaining the button in a compressed position.

20. The catheter system of claim 1, wherein the button comprises one or more channels that are formed perpendicular to a length of the button.
